(19) United States
(12) Patent Application Publication
(10) Pub. No.: US 2017/0292940 A1
(43) Pub. Date: Oct. 12, 2017

(54) Computerized Fluid Analysis for Determining Whether an Asset is Likely to Have a Fluid Issue

(71) Applicant: Uptake Technologies, Inc., Chicago, IL (US)

(72) Inventors: Aparna Pandey, Chicago, IL (US); Michael Horrell, Chicago, IL (US); Alexander Gutfraind, Chicago, IL (US)

(21) Appl. No.: 15/092,593

(22) Filed: Apr. 6, 2016

(51) Int. Cl. G01N 33/28

(52) U.S. Cl. CPC G01N 33/2888

(57) ABSTRACT

Disclosed herein are systems, devices, and methods related to a determination of whether an asset has a fluid issue. In particular, examples involve a platform defining a predictive model for outputting an indicator of whether an asset is likely to have a fluid issue based at least on historical fluid data for one or more assets. The historical fluid data may comprise at least one of a plurality of fluid reports for the one or more assets and an indication of a fluid issue for each fluid report. The platform may receive at least one fluid report associated with a given asset and based at least on the predictive model and the received at least one fluid report, make a determination that the given asset is likely to have a fluid issue. The platform may cause a computing device to output an indication of the determination.

### BACKGROUND

Today, assets (also referred to herein as “machines”) are ubiquitous in many industries. From locomotives that transfer cargo across countries to medical equipment that helps nurses and doctors to save lives, assets serve an important role in everyday life. Depending on the role that the asset serves, its complexity may vary. For instance, some assets may include multiple subsystems that must operate in harmony for the asset to function properly (e.g., an engine, transmission, etc. of a locomotive).

Fluid analysis is one mechanism for making sure that an asset is operating properly. Assets, such as mining equipment, locomotives, aircrafts, and ships, commonly use fluid to operate. The fluid may take the form of oil, transmission, coolant, or fuel, for example. The fluid travels through many subsystems in the asset to perform various functions, including cooling, hydraulic, and lubrication functions. In this process, the fluid may be exposed to harsh environments, including heat, pressure, and friction, which results in properties of the fluid changing. For instance, oil may pick up particles associated with the wearing of asset components that the oil is lubricating. The particles may include, for example, aluminum particles from an aluminum piston, aluminum particles from an aluminum piston liner, or contamination from the environment. Some changes in fluid composition may be expected due to normal operation of the asset. Other changes, however, may be suggestive of an abnormal operation. The fluid analysis is a determination of the properties of the fluid in the asset. The properties can be evaluated to determine whether the asset is operating properly and to assist in performing preventative maintenance.

### OVERVIEW

A fluid analyzer is a device which analyzes fluid from an asset and determines the properties of the fluid. These properties may include information regarding particles in the fluid such as particle types, particle counts, and particle sizes. A particle may be a constituent of the fluid, which may take the form of solids, liquids, and gases. An example of a particle in solid form may be a piece of metal such as aluminum, an example of a particle in liquid form may be water or diesel, and an example of a particle in gaseous form may be hydrogen or carbon dioxide. The particle type may indicate a particular type of particle in the fluid, a particle count may indicate an amount or concentration of the type of particle in the fluid, and the particle size may indicate a size of the particles in the fluid. The fluid analyzer could determine other properties of the fluid as well, such as pH and viscosity of the fluid.

During a fluid analysis, the fluid analyzer may check for tens, hundreds, or even thousands of different particles in the fluid, including presence or absence of certain elements and compounds that exist in the asset itself or in the environment in which the asset is operating. The fluid analyzer then typically outputs its findings in a complex fluid report which may specify in detail the particle type, particle counts, particle sizes for all of the different particles that are found. Additionally, the fluid report may specify the pH and viscosity of the fluid, among other properties.

The fluid may have some expected properties, due to the inherent nature of the fluid or from normal operation of the asset. But other properties may be indicative of an abnormality in the operation of the asset (a failure or potential failure of a component in the asset that may require attention), in which case the asset may be deemed to have a “fluid issue.” Examples of a fluid issue with the asset or a specific subsystem of an asset may be due to wear and tear of a component, presence of contaminants, or leaks in fluid that may lead to damage to the asset etc. For instance, if a high concentration of aluminum particles is found in a fluid, then this may indicate a failure or imminent failure of an aluminum component in the asset. Further, type of fluid can help localize the specific component in the asset that is failing or about to fail. For example, if the fluid is engine oil, then the aluminum component that may be failing or about to fail may be the aluminum piston liner or the aluminum piston which is lubricated by the engine oil.

The example above is merely provided for illustration, and is an oversimplification of the contents and use of the fluid report. In practice, making a determination of whether the asset has a fluid issue is an extremely difficult process. The determination is hindered by the sheer number of properties analyzed by the fluid analyzer and indicated in the fluid report. Indeed, as noted above, a fluid analyzer typically outputs a complex report that includes a wide range of different properties measured by the fluid analyzer, and whether these properties are indicative of a fluid issue may depend on various other factors, such as the type of fluid, the type of asset, and/or the subsystem of the asset from which the fluid was taken. Moreover, not just one fluid report but a sequence of fluid reports (showing changes or trends in the types and amounts of the particles in the fluid over time) could be used in the determination of a fluid issue. The example systems, devices, and methods disclosed herein seek to help address one or more of these difficulties in analyzing the complex fluid reports generated by the fluid analyzer and making reliable determinations of whether an asset has a fluid issue.

In general, a platform may be configured to obtain historical fluid data, analyze the historical fluid data, and define a predictive model which correlates properties of the fluid to an indication, e.g., probability, of an asset having a fluid issue.

The historical fluid data may be at least one of a plurality of fluid reports generated by a fluid analyzer and an indication for each of the fluid reports of whether an asset associated with the fluid report has a fluid issue. The plurality of fluid reports may include an analysis of a sample of the fluid recently taken from the asset as well as an analysis of samples of the fluid taken from the asset at some earlier time(s). This way the historical fluid data may maintain a history of the properties of the fluid and changes in the properties of the fluid associated with the asset over time.

The historical fluid data may include fluid reports generated from an analysis of a same type of fluid (e.g., oil or coolant fluid). As another example, the fluid reports may be generated from an analysis of the fluid reports of a same type of fluid from a same type of asset (e.g., an airplane or a tractor). As yet another example, the fluid reports may be generated from an analysis of fluid reports of a same type of fluid from a same type of subsystem of an asset (e.g., an engine or transmission). In another example, the fluid reports may be generated from an analysis of fluid reports of a same type of fluid from the same asset itself.

The platform may receive this historical fluid data and define a predictive model based on an analysis of relationships between (1) properties of a fluid report and (2) whether there is a fluid issue. The platform may then use the predictive model to predict a likelihood of a fluid issue at a given asset based on a fluid report. For instance, the platform may receive recent fluid report(s) for a fluid associated with a given asset and then input the fluid report(s) into the predictive model. In turn, the predictive model may output an indicator of whether the given asset is likely to have a fluid issue. Additionally, the platform may also use the model to identify which properties of the fluid are contributing to the asset's likelihood of having the fluid issue.

The platform may then take various action based on the output of the model. For instance, in one implementation, the platform may generate an alert message based on the output of the predictive model (e.g., if the likelihood of the asset having a fluid issue exceeds some threshold amount). The alert may be a visual and/or audible to a user on a client station such as a computer or personal device like a mobile phone, tablet, and so on.

In another implementation, based on the output of the model, the platform may trigger various types of preventative actions. For example, based on the probability, the platform may facilitate generating a work order to repair the asset with fields populated based on the results of the predictive model to facilitate the workflow of the user. These fields could include but are not limited to suggested repairs, suggested preventative maintenance procedures and so on. In another embodiment, the client station may also show the detailed instructions for suggested repairs or procedures. In other examples, the platform may facilitate ordering a part to repair the asset, and/or transmit to the asset one or more commands that cause the asset to modify its operation. Other arrangements are also possible.

Additionally, the platform 110 may visually present information associated with the fluid report or plurality of fluid reports. The information may be presented as a graphical user interface (GUI) on the client station 112. For instance, in one implementation, the GUI may include a textual or graphical representation of particles counts for the various particles in a given type of fluid taken from a given asset. In another implementation, the GUI may include a textual and/or graphical presentation of how certain properties of the given type of fluid taken from the given asset compare to properties of the given type of fluid taken from other assets. This information may be presented in the form of numerical data, a bar graph, or line graph on the GUI.

In one aspect, for instance, a method comprises defining a predictive model for outputting an indicator of whether an asset is likely to have a fluid issue. The predictive model is based at least on historical fluid data for one or more assets. The historical fluid data comprises at least one of (i) a plurality of fluid reports associated with the one or more assets and (ii) a plurality of indications and each indication identifies whether an asset of the one or more assets has a fluid issue. At least one fluid report associated with a given asset is received and based at least on the predictive model and the received at least one fluid report, a determination is made that the given asset is likely to have a fluid issue; and a computing device is caused to output an indication of the determination.

The indication of a fluid issue is determined based on an expert review of the fluid report. Defining the predictive model comprises applying a regression technique or classification to the historical fluid data. The historical fluid data comprises one or more of (i) a plurality of fluid reports for a same type of fluid; (ii) a plurality of fluid reports for a same type of the given asset; (iii) a plurality of fluid reports for the given asset; and (iv) a plurality of fluid reports for a same type of subsystem of the given asset. Each of the fluid reports comprises a plurality of fluid properties. The fluid properties comprise a particle count of particles in a fluid and other properties such as the age of the fluid, observed viscosity and so on. Each fluid report is for a given type of fluid taken from the given asset. The plurality of fluid reports comprises a sequence of fluid reports for the given asset. Causing a computing device to output an indication of the determination comprises causing a computing device to display a visual indication of the determination. The determination that the given asset is likely to have a fluid issue is based at least on the predictive model.

The received at least one fluid report comprises: applying the predictive model to the received at least one fluid report to output an indicator of whether the given asset is likely to have a fluid issue; comparing the indicator to a threshold condition; and determining that the indicator exceeds the threshold condition. The computing device is further caused to output an indication of those properties of a given type of fluid of the given asset which influence the determination that the given asset is likely to have the fluid issue. The received at least one fluid report defines a plurality of properties of a fluid and a measure associated with each property. The method further comprises selecting a given property of the received at least one fluid report; changing a given measure of the given property in the at least one fluid report; making a determination that changing the given measure causes a probability that the given asset has the fluid issue to change by a threshold amount; and causing the computing device to output an indication of the given property.

In another aspect, a platform comprises: a network interface configured to facilitate communication with a data source and a computing device via a communication network; at least one processor; a non-transitory computer-readable medium; and program instructions stored on the non-transitory computer-readable medium that are executable by the at least one processor to cause the platform to: receive at least one fluid report associated with a given asset; based at least on a predictive model which outputs an indicator of whether an asset is likely to have a fluid issue and the received at least one fluid report, make a determination that the given asset is likely to have a fluid issue, wherein the predictive model is defined based at least on historical fluid data for one or more assets received from the data source, wherein the historical fluid data comprises at least one of (i) a plurality of fluid reports associated with the one or more assets and (ii) a plurality of indications, wherein each indication identifies whether an asset of the one or more assets has a fluid issue; and cause the computing device to output an indication of the determination.

The plurality of fluid reports comprises a sequence of fluid reports for the given asset. The program instructions for causing a computing device to output an indication of the determination comprises causing the computing device to display a visual indication of the determination. The historical fluid data comprises one or more of (i) a plurality of fluid reports for a same type of fluid; (ii) a plurality of fluid reports for a same type of the given asset; (iii) a plurality of fluid reports for the given asset; and (iv) a plurality of fluid reports for a same type of subsystem of the given asset. The program instructions for making a determination that the given asset is likely to have a fluid issue based at least on the predictive model and the received at least one fluid report comprises program instructions for: applying the predictive model to the received at least one fluid report to output an indicator of whether the given asset is likely to have a fluid issue; comparing the indicator to a threshold condition; and determining that the indicator exceeds the threshold condition. Program instructions further cause the computing device to output an indication of how properties of a given type of fluid in the given asset compare to properties of the given type of fluid in other assets based on the historical fluid data. The received at least one fluid report defines a plurality of properties of a fluid and a measure associated with each property, the platform further comprising program instructions for: selecting a given property of the received at least one fluid report; changing a given measure of the given property in the at least one fluid report; making a determination that changing the given measure causes a probability that the given asset has the fluid issue to change by a threshold amount; and causing the computing device to output an indication of the given property.

In yet another aspect, a non-transitory computer-readable medium having instructions stored thereon that are executable to cause a platform to: receive at least one fluid report associated with a given asset; based at least on a predictive model which outputs an indicator of whether an asset is likely to have a fluid issue and the received at least one fluid report, make a determination that the given asset is likely to have a fluid issue, wherein the predictive model is defined based at least on historical fluid data for one or more assets received from a data source, wherein the historical fluid data comprises at least one of (i) a plurality of fluid reports associated with the one or more assets and (ii) a plurality of indications, wherein each indication identifies whether an asset of the one or more assets has a fluid issue; cause a computing device to output an indication of the determination.

One of ordinary skill in the art will appreciate these as well as numerous other aspects in reading the following disclosure

### BRIEF DESCRIPTION OF THE DRAWINGS

FIG. 1 depicts an example network configuration in which example embodiments may be implemented.

FIG. 2 illustrates an example excerpt of a fluid report.

FIG. 3 is a structural block diagram of an example platform.

FIG. 4 is a flow diagram of example functions performed by the example platform to determine whether an asset has a fluid issue.

FIG. 5 is a flow diagram of example functions performed by the example platform during a modeling phase.

FIG. 6 is a flow diagram of example functions performed by the example platform during an asset monitoring phase.

FIG. 7 is a flow diagram of example functions performed by the example platform to identify properties of a fluid influential on a probability of an asset having a fluid issue.

FIG. 8 illustrates an example user interface of a client station.

FIG. 9 is a flow diagram of example functions performed by the example platform.

### DETAILED DESCRIPTION

The following disclosure makes reference to the accompanying figures and several exemplary scenarios. One of ordinary skill in the art will understand that such references are for the purpose of explanation only and are therefore not meant to be limiting. Part or all of the disclosed systems, devices, and methods may be rearranged, combined, added to, and/or removed in a variety of manners, each of which may be contemplated herein.

### I. Example System Configuration

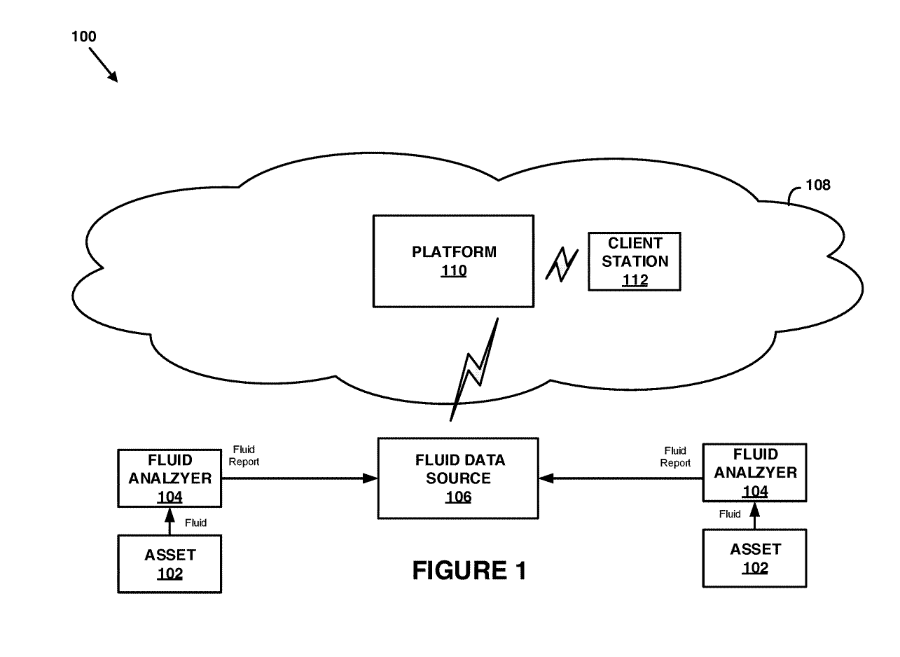

FIG. 1 illustrates an example system configuration 100 for predicting whether an asset has a fluid issue based on a fluid report generated by a fluid analyzer. The system configuration may include an asset 102, a fluid analyzer 104, a fluid data source 106, a communication network 108, a platform 110, and a client station 112. The system configuration may include a plurality of assets and plurality of fluid analyzers. Additionally, in some embodiments, the system configuration may also include a plurality of platforms and respective client stations that perform the functionality described herein.

This example network configuration is illustrated in context of asset management. However, it should be understood that the concepts disclosed herein may be applied in any other context outside of asset management where parties and other organizations have an interest managing assets based on fluid analysis.

In general, the communication network 108 may include one or more computing systems and network infrastructure configured to facilitate transferring data between the fluid data source 106, the platform 110, and client station 112. The communication network 108 may be or may include one or more Wide-Area Networks (WANs) and/or Local-Area Networks (LANs), Personal Area Network (PAN) and/or Vehicular Area Network (VAN) and/or ad hoc networks, and/or wired and/or wireless networks. In some examples, the communication network 108 may include one or more cellular networks and/or the Internet, among other networks. The communication network 104 may operate according to one or more communication protocols, such as LTE, CDMA, WiMax, WiFi, Bluetooth, HTTP, TCP, and the like. Although the communication network 108 is shown as a single network, it should be understood that the communication network 108 may include multiple, distinct networks that are themselves communicatively linked.

Each asset 102 can take a variety of forms, examples of which may include transportation machines (e.g., locomotives, aircrafts, semi-trailer trucks, ships, etc.), industrial machines (e.g., mining equipment, construction equipment, etc.), medical machines (e.g., medical imaging equipment, surgical equipment, medical monitoring systems, medical laboratory equipment, etc.), and utility machines (e.g., turbines, solar farms, etc.), among other examples. The assets may be composed of various subsystems including engines, transmissions, coolant systems, and fuel systems that enable the asset to perform a function. Additionally, assets of a given type may have various different configurations depending on brand, make, model, etc. Those of ordinary skill in the art will appreciate that these are but a few examples of assets and that numerous other examples are possible and contemplated herein.

A common thread among each of the assets types is that they each have one or more subsystems that require fluid to operate. These subsystems may include, but are not limited, engine, hydraulic, transmission, fuel, and coolant subsystems. The fluid may take a variety of forms including oil, fuel, hydraulic fluid, brake fluid, coolant, and the sort. The disclosed embodiments are not limited by the type of asset or the type of fluid used by the asset.

The fluid analyzer 104 is a machine which analyzes fluid from an asset to determine the various properties of the fluid. The properties may include a particle analysis of the fluid such as identification of particle types in the fluid, particle counts in the fluid, and particle sizes in the fluid. A particle may represent a constituent of the fluid, which may take the form of solids, liquids, and gases. An example of a particle in solid form may be a metal such as aluminum, an example of a particle in liquid form may be a water or diesel, and an example of a particle in gaseous form may be hydrogen or carbon dioxide. The particle type may be an identification of a particular type of particle in the fluid, a particle count may indicate an amount or concentration of the type of particle in the fluid, and the particle size may indicate a range of sizes of the particles in the fluid. The fluid analyzer may determine other properties of the fluid as well, such as pH and viscosity of the fluid.

The fluid may be provided to the fluid analyzer 104 in many different ways. In one example, a sample of the fluid may be physically removed from the asset 102 and placed in the fluid analyzer 104 for analysis. In another example, the fluid analyzer 104 may be onboard the asset 102 and the asset 102 may have one or more testing equipment, such as sensors or probes in the fluid. The testing equipment may enable the fluid analyzer 104 to sample and analyze the fluid directly in the asset without needing to be physically removed from the asset. Other arrangements are also possible.

The fluid analyzer 104 may perform a variety of tests on the fluid to determine the properties of the fluid. The tests may include, but are not limited to, a chemistry test, turbidity test, infrared test, conductivity test, spectrometer test, composition test, viscosity test, among others to determine the properties of the fluid. The fluid analyzer 104 may test for tens, hundreds, or thousands of different particles of the fluid, including presence or absence of certain elements and compounds found in the asset itself or in the environment in which the asset is operating. Examples of fluid analyzers for analyzing asset fluids include the Spectro-Scientific FluidScan Q1000 and Maersk Fluid Technology's SEA-Mate Analyzer.

The fluid analyzer 104 may output its findings in a complex fluid report that indicates various properties of the fluid. The fluid report may specify in detail the particle type, particle counts, particle sizes for all of the different properties that are tested. Additionally, the fluid report may specify the pH and viscosity of the fluid, among other properties.

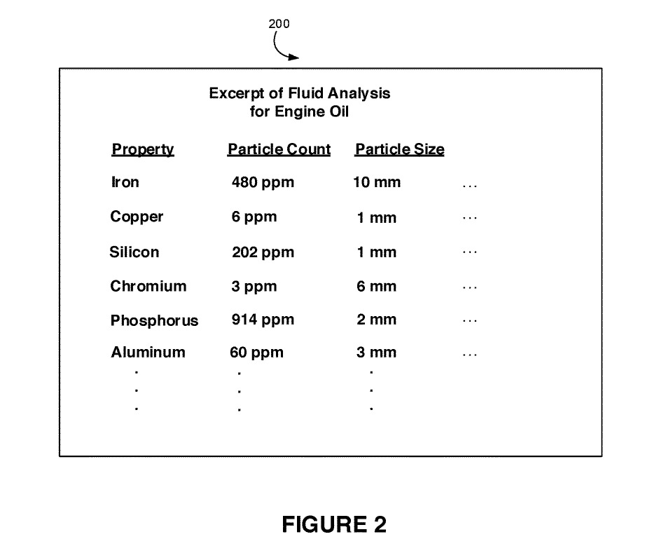

FIG. 2 is an example excerpt of one such fluid report for engine oil. The example fluid report illustrates just a fraction of the properties that can be measured by the fluid analyzer 104. The fluid report identifies properties of the fluid, such as particle counts and particle sizes for various types of particles in the fluid, examples of which may include iron, copper, silicon, chromium, phosphorous, and aluminum. The particle counts may be measured in parts per million (ppm as shown in FIG. 2) or as percentage of the composition of the fluid and the particle sizes may be measured in millimeters (mm), for example. Some fluid elements and compounds may also be measured in quantized categories such as presence in “Low” or “Medium” or “High” amounts. The fluid report may be output in the form of an electronic file such as a data file, email, or message, which may be stored, downloaded, accessed, or transmitted to another location remote from the fluid analyzer 104.

In some embodiments, the fluid in the asset may additionally or alternatively be physically inspected by a human to assess certain properties of the fluid. The properties observable by physical inspection may include, but is not limited, to odor, appearance, and texture of the fluid. In the case of a physical inspection, an individual skilled in the art such as a laboratory technician, a fluid interpreter and such may document the properties of the fluid in a fluid report. The properties may be recorded in the fluid report along with or instead of the properties identified by the fluid analyzer 104.

Referring back to FIG. 1, a fluid report associated with an asset may be provided to a fluid data source 106, which may include one or more computing systems configured to receive fluid reports from the fluid analyzer 104, aggregate the fluid reports from a plurality of fluid analyzers, and store the fluid reports. The fluid reports may be stored in the fluid data source 106 as one or more data records defining the properties of a fluid.

The fluid data source 106 may also provide an indication of whether the asset has a fluid issue. The indication of the fluid issue may be determined by an expert trained in fluid analysis who may analyze the properties of a fluid report to determine whether the asset has a fluid issue. The expert may consider the type of particles present in the fluid, the particle count, the particle sizes, the type of the fluid, the type of the asset, the age of the asset, the change in properties of a fluid in a sequence of fluid reports associated with fluid analysis conducted over a period of time, among other factors to determine whether the asset has a fluid issue. For example, the expert may review the fluid report and determine that an amount of iron exceeding 450 ppm in the engine oil along with an amount of silicon exceeding 200 ppm may suggest a fluid issue. As another example, the expert may review the fluid report and determine that an amount of aluminum exceeding 100 ppm in the engine oil and an amount of iron less than 100 ppm may suggest a fluid issue. In this regard, the expert may use the properties of the fluid, as indicated by the fluid report, to determine whether the asset has a fluid issue.

The example above is merely provided for illustration, and is an oversimplification of the contents and use of the fluid report. In practice, making a determination of whether the asset has a fluid issue based on a fluid report (or sequence of fluid reports) is an extremely difficult process. The determination is hindered by the sheer number of properties analyzed by the fluid analyzer and indicated in the fluid report. Indeed, as noted above, a fluid analyzer typically outputs a complex report which includes a wide range of different properties measured by the fluid analyzer, and whether these properties are indicative of a fluid issue may depend on various other factors, such as the type of fluid, the type of asset, the subsystem of the asset from which the fluid was taken. Moreover, not just one but a sequence of fluid reports (showing changes in the types and amounts of the particles in the fluid over time) could be used in the determination of a fluid issue.

An expert trained in the field of fluid analysis and familiar with the type of asset which sourced the fluid may spend hours studying a plurality of fluid reports associated with an asset to determine whether the asset has a fluid issue. The plurality of fluid reports can include the analysis conducted on a recent fluid sample taken from the asset as well as the analysis conducted on fluid samples previously taken from the asset. The expert may analyze one or more of these fluid reports to identify the properties in a particular fluid report indicative of a fluid issue. Additionally, the expert may analyze changes in the properties of the fluid over time as indicated by the fluid reports associated with the recent fluid samples and the fluid samples previously taken from the asset (e.g., a sequence of fluid reports). Moreover, there is even no guarantee that a fluid issue may be correctly identified. Identification of the fluid issue is heavily dependent on the specific training of the expert performing the analysis.

The expert may input into fluid data source 106 his or her determination of whether there is a fluid issue for an asset associated with a fluid report. For example, the expert may input his or her determination via a user interface coupled to the fluid data source 106. The fluid data source 106 may associate the fluid report(s) that the expert reviewed with the determination of whether there is a fluid issue. For example, the determination of whether there is a fluid issue may be stored in a data record defining the properties of the fluid or in some other data record.

The system configuration 100 may include tens or hundreds of assets and fluid analyzers, and as a result, tens, hundreds, or even thousands of fluid reports may be associated with the plurality of assets. In this regard, a plurality of experts may be reviewing fluid reports and inputting the determination of whether there is a fluid issue for a fluid report associated with an asset. Each expert may be reviewing fluid reports and inputting determinations of a fluid issue for a fluid report which are stored by the fluid data source 106.

Accordingly, in addition to determining fluid issues, the expert may also validate determinations of fluid issues associated with the fluid reports stored by the fluid data source 106. The determination of the fluid issue may have been made by an expert or using the predictive model described in further detail below. The expert may review the fluid report or sequence of fluid reports and confirm agreement with the determination of the fluid issue made by another expert or the predictive model. The expert may input his or her agreement to the determination via the user interface of the fluid data source 106.

The embodiment described above involves an expert providing the determination of the fluid issue. However, in some embodiments, instead of the expert providing the determination of a fluid issue, a computer system might provide the determination of the fluid issue. For example, a sensor on an asset may be configured to detect physical properties of the asset, and provide indications, such as electrical signals, associated with the detected physical properties. Examples of physical properties may include, but is not limited, to temperatures, pressures, speeds, acceleration or deceleration rates, friction, power usages, fuel usages, fluid levels, runtimes, voltages and currents, magnetic fields, electric fields, presence or absence of objects, positions of components, and power generation, among other examples. The computer system may process these indications, to determine whether the fluid has a fluid issue. No expert might be involved in this determination or the expert may use a computer system in making the determination. Other arrangements are also possible.

The system configuration 100 may have a platform 110 (referred to herein also as a computer system). Broadly speaking, the platform 110 may take the form of one or more computer systems that are configured to receive and analyze asset-related data, e.g., fluid reports, for one or more assets. For instance, the platform 110 may include one or more servers (or the like) having hardware components and software components that are configured to carry out one or more of the functions disclosed herein. In practice, these computing systems may be located in a single physical location or distributed amongst a plurality of locations, and may be communicatively linked via a system bus, a communication network (e.g., a private network), or some other connection mechanism. Furthermore, the physical location of the platform 110 can be fixed or mobile.

The communication network 108 may communicatively couple the platform 110 and fluid data source 106. The platform 110 may receive historical fluid data from the fluid data source 106. The historical fluid data may refer to at least one of the plurality of fluid reports generated by one or more fluid analyzers and associated indications for each fluid report of whether there is a fluid issue for an asset associated with the fluid report. The plurality of fluid reports may include an analysis of recent samples of the fluid of an asset as well as analysis of previous samples of the fluid of the asset (e.g., a sequence of fluid reports). This way the historical fluid data may maintain a history of the properties of the fluid for the asset over time. The historical fluid data may be further defined by fluid reports generated from an analysis of a same type of fluid (e.g., oil or coolant fluid). As another example, the fluid reports may be generated from an analysis of the fluid reports of a same type of fluid from a same type of asset (e.g., an airplane or a tractor). As yet another example, the fluid reports may be generated from an analysis of fluid reports of a same type of fluid from a same type of subsystem of an asset (e.g., an engine or transmission). In another example, the fluid reports may be generated from an analysis of fluid reports of a same type of fluid from a specific asset.

The platform 110 may be configured to obtain the historical fluid data and define based on the fluid reports of the historical fluid data a predictive model which correlates properties of a fluid to an indication of whether the asset has a fluid issue. Thereafter, the platform 110 may receive a new fluid report associated with a given asset, where it is not known based on the fluid report whether or not the asset has a fluid issue, input the fluid report into the model, and predict whether the given asset has a fluid issue. In this regard, the platform 110 may take as input the fluid report and the model may output an indicator of whether the asset is likely to have a fluid issue.

Additionally, a client station 112 may be associated with the platform 110. The client station 112 may take the form of a computing system or device configured to receive input, process data, and provide an output. Examples of client stations 112 include a single or network of multiple connected desktop computers, storage devices, tablets, smartphones, laptop computers, other mobile computing devices, smart TVs, wearable devices, and the like. In one example, one or more of the client stations 112 may be or include one or more input devices configured to receive data and one or more output devices configured to provide audible, visual, and/or tactile output in response to the data. In general, an input device may include one or more input interfaces configured to receive user input, and can include a keyboard, microphone, pointing device (mouse, trackball, etc.), camera, sensors, etc. An output device may be configured to provide output to a user and/or to transmit data through the communication network 108 based on such user input, and can include a display device (display screen, projector, goggles, etc.), printing device, haptic output device, etc.

The platform 110 may cause one or more actions to be taken on the client station 112 based on the output of the model. For instance, in one implementation, the platform 110 may generate an alert message based on the output of the predictive model (e.g., if the likelihood of the asset having a fluid issue exceeds some threshold amount) on the client station 112. The alert may be visual and/or audible to a user of the client station.

In another implementation, based on the output of the model, the platform 110 may trigger various types of preventative actions. For example, based on the probability, the platform 110 may facilitate generating a work order to repair the asset, facilitate ordering a part to repair the asset, and/or transmit to the asset one or more commands that cause the asset to modify its operation. Other preventative actions are also possible.

Additionally, or alternatively, the platform 110 may cause a graphical user interface (GUI) to be presented on the client station 112 with information associated with a fluid report or a plurality of fluid reports. For instance, in one implementation, the GUI may include a textual or graphical representation of particles counts for the various particles in a given type of fluid taken from a given asset. In another implementation, the GUI may include a textual and/or graphical presentation of how certain properties of the given type of fluid taken from the given asset compare to properties of the given type of fluid taken from other assets. This information may be presented in the form of numerical data and/or one or more types of graphs such as bar graphs, line graphs, or bubble charts on the GUI. In yet another implementation, the client station 112 may show the work order form with fields populated based on the results of the predictive model to facilitate a workflow for the user. These fields could include but are not limited to suggested repairs, suggested preventative maintenance procedures and so on. Additionally, the client station 112 may also show detailed instructions for suggested repairs or procedures.

### II. Example Platform

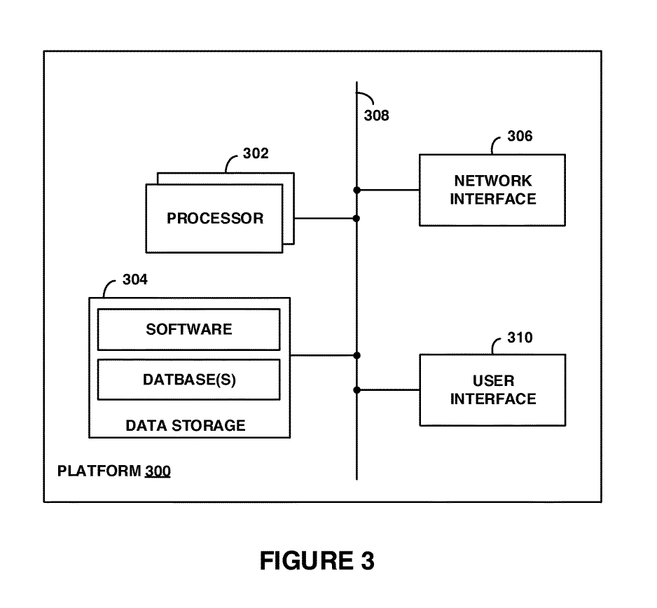

Referring now to FIG. 3, a simplified block diagram illustrating some components in the example platform 300 from a structural perspective is depicted. As noted above, a platform 300 may generally comprise one or more computer systems (e.g., one or more servers), and these one or more computer systems may collectively include a processing unit 302, data storage 304, network interface 306, communication link 308, and perhaps also a user interface 310. One of ordinary skill in the art will appreciate that the platform 300 may include additional components not shown and/or more or less of the depicted components.

The network interface 306 may be configured to facilitate wireless and/or wired communication between the platform 300 and various network components coupled to the communication network 108, such as the fluid data source 106. As such, network interface 306 may take any suitable form for carrying out these functions, examples of which may include an Ethernet interface, a serial bus interface (e.g., Firewire, USB 2.0, etc.), a chipset and antenna adapted to facilitate wireless communication, and/or any other interface that provides for wired and/or wireless communication. Network interface 306 may also include multiple network interfaces that support various different types of network connections to systems related to data storage, computational needs and data transmission, some examples of which may include Hadoop, FTP, relational databases, high frequency data such as OSI PI, batch data such as XML, and Base64. Other configurations are possible as well.

The processor 302 may include one or more processors and/or controllers, which may take the form of a general or special-purpose processor or controller. In particular, in example implementations, the processing unit 302 may include microprocessors, microcontrollers, application-specific integrated circuits, digital signal processors, and the like.

In turn, data storage 304 may comprise one or more non-transitory computer-readable storage mediums, examples of which may include volatile storage mediums such as random access memory, registers, cache, etc. and non-volatile storage mediums such as read-only memory, a hard-disk drive, a solid-state drive, flash memory, an optical-storage device, etc.

As shown in FIG. 3, the data storage 304 may be provisioned with software components that enable the platform 300 to carry out the functions disclosed herein, including defining the model and applying the model to determine whether an asset has a fluid issue. These software components may generally take the form of program instructions that are executable by the processor 302, and may be arranged together into applications, software development kits, toolsets, or the like. In addition, the data storage 304 may also be provisioned with one or more databases that are arranged to store data related to the functions carried out by the platform, examples of which include time-series databases, document databases, relational databases (e.g., MySQL), key-value databases, and graph databases, among others.

In some embodiments, the example platform 300 may support a user interface 310 that is configured to facilitate user interaction with the platform 110 and may also be configured to facilitate causing the platform 110 to perform an operation in response to user interaction. This user interface 310 may include or provide connectivity to various input components, examples of which include touch-sensitive interfaces, mechanical interfaces (e.g., levers, buttons, wheels, dials, keyboards, etc.), and other input interfaces (e.g., microphones). Additionally, the user interface 310 may include or provide connectivity to various output components, examples of which may include display screens, speakers, headphone jacks, and the like. Other configurations are possible as well.

### III. Example Operations

The configurations depicted in FIGS. 1 and 3 will now be discussed in further detail below. To help describe some of these operations, functional block diagrams may be referenced. In some cases, each block may represent a module or portion of program code that includes instructions that are executable by a processor to implement specific logical functions or steps in a process. The program code may be stored on any type of computer-readable medium, such as non-transitory computer-readable media. In other cases, each block may represent circuitry that is wired to perform specific logical functions or steps in a process. Moreover, the blocks shown in the flow diagrams may be rearranged into different orders, combined into fewer blocks, separated into additional blocks, and/or removed based upon the particular embodiment.

The following description may further reference examples of using one or more fluid reports associated with a same type of fluid to define a model. The model is, in turn, used to determine whether an asset has a fluid issue. It should be understood that this is done merely for sake of clarity and explanation and is not meant to be limiting.

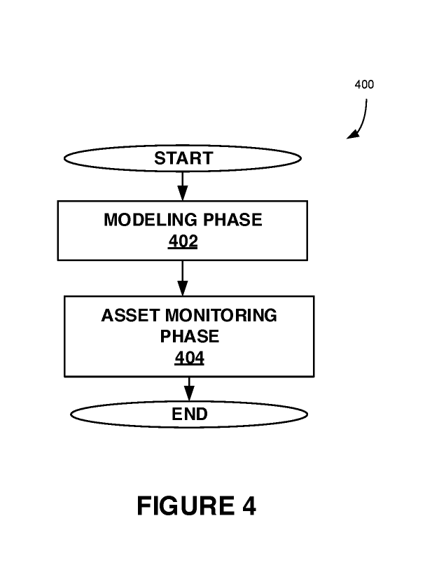

FIG. 4 illustrates two phases in the determination as to whether an asset has a fluid issue based on a fluid report. The two phases are referred to as a “modeling phase” and an “asset-monitoring phase.” Each phase may be performed, in part, by the platform 110.

The “modeling phase” at 402 may include defining a model based on historical fluid data for predicting whether an asset has a fluid issue. In general, the model may correlate properties of the fluid to an indication of whether the asset has a fluid issue. The historical fluid data may include a plurality of fluid reports generated by a fluid analyzer 104 for a plurality of fluids. The historical fluid data may also indicate for each fluid report whether the asset has a fluid issue. The platform 110 may receive this historical fluid data from the fluid data source 106. Based on this data, the platform 110 may be configured to define a predictive model (also referred to herein generally as model) which correlates the properties of a fluid report to an indication of whether the asset has a fluid issue.

Then, during the “asset-monitoring” phase, at 404, the platform 110 may apply the predictive model defined in the modeling phase to determine whether a given asset has a fluid issue. During the asset-monitoring phase, the platform 110 may receive a fluid report of a fluid from the fluid data source 106. The fluid report may not have any indication of whether there is a fluid issue associated with the asset. The platform 110 may use the predictive model to determine a likelihood of the given asset having a fluid issue.

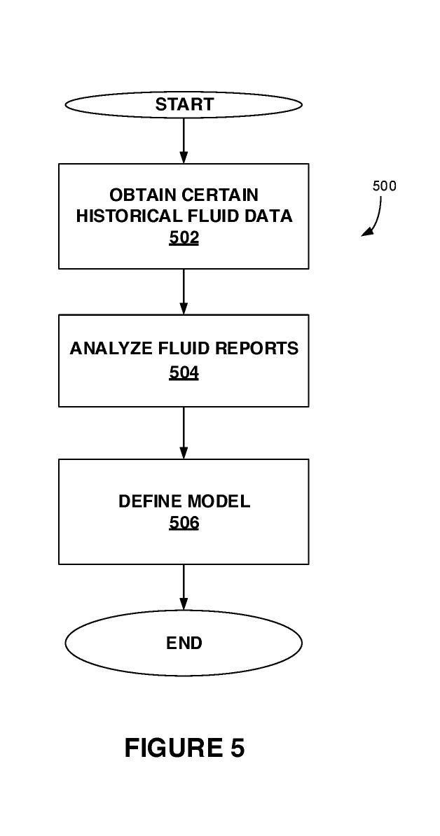

FIG. 5 is a flow diagram 500 depicting one possible example of the modeling phase. For purposes of illustration, the example modeling phase is described as being carried out by the platform 110. One of ordinary skill in the art will appreciate that the flow diagram 500 is provided for sake of clarity and explanation. Numerous other combinations of operations may be utilized in determining the model including, but not limited to, the modeling phase being performed offline with human intervention.

As shown in FIG. 5, at 502, the platform 110 may obtain certain historical fluid data. At 504, the platform 110 may analyze the fluid reports to define relationships between (1) properties of a fluid report and (2) whether the asset associated with the fluid report has a fluid issue. Lastly, at 506, the relationships may be combined to define a model for predicting a likelihood of an asset having a fluid issue based on a fluid report (or sequence of fluid reports).

The fluid data source 106 may continue to receive fluid reports and indications of whether the asset has a fluid issue based on the fluid reports. This historical data can then be used by the platform 110 to continue to refine the model by repeating functions in blocks 502-506.

The functions of the example modeling phase illustrated in FIG. 5 will now be described in further detail.

Starting at 502, the platform 110 may obtain from the fluid data source 106 certain historical fluid data stored in the fluid data source 106. The historical fluid data may be a plurality of fluid reports generated by a fluid analyzer 104. The historical fluid data may also include an indication of whether the asset associated with the fluid report has a fluid issue.

In some embodiments, this indication may be a binary (i.e., yes/no) decision as to whether the asset has a fluid issue. In this regard, the historical fluid data may include fluid reports which indicate that an asset has a fluid issue and others which do not indicate that an asset has a fluid issue. Moreover, each fluid report may have been analyzed the same expert or different experts who each used different criteria in concluding whether the asset has a fluid issue.

In some embodiments, a fluid issue may be a multi-class indication, for example representing multiple levels of fluid issues, rather than simply the presence of a fluid issue. For example, the fluid issue may be encoded as a plurality of levels, such as a “High”, “Medium”, “Low” that indicate a severity of the fluid issue in contrast to no fluid issue. As an another example, the fluid issue may be an indication of a specific type of fluid issues such as “Contamination”, “Leak”, again in contrast to no fluid issue. Other arrangements are also possible.

The historical fluid data and/or the fluid reports themselves may have various identifying data. The identifying data may include a timestamp indicating when a fluid analysis was performed or a timestamp indicating the date/time when a fluid of the asset was sampled. The historical fluid data may also identify a type of fluid analyzed. The identification of the type of fluid may be a general category such as engine oil or be a specific identification such as Pennzoil SAE 5 W-30 engine oil. Additionally, the historical fluid data may identify the asset which sourced the fluid. Again, the identification may be general such as an identification of a type of asset which sourced the fluid (e.g., tractor or airplane) and/or the component of the asset which sourced the fluid (e.g., engine or transmission) or specific such as the asset with Serial No. 112. Other identification is also possible.

The historical fluid data may be associated with one or more of a same type of fluid, such as engine oil or coolant fluid. As another example, the historical fluid data may be associated with a particular asset, such as the asset with a particular serial number. As yet another example, the historical fluid data may be associated with a same type of asset. The same type of asset may be those from a same manufacturer(s), with a same model number(s), or those used in similar environments or operating conditions. As another example, the historical fluid data may be associated with same type of subsystem such as an engine or transmission.

In some embodiments, the platform 110 may request from the fluid data source 106 certain historical fluid data and the platform 110 may receive from the fluid data source 106 the requested historical fluid data. For example, the platform 110 may request historical fluid data for a type of fluid, for fluid from a particular asset, or for fluid associated with a subsystem of an asset. Additionally, or alternatively, the platform 110 may request a particular range of historical fluid data. For example, the platform 110 may identify a certain amount of time's worth of data. The time could be those fluid reports for fluid sampled in a window of time and/or those fluid reports analyzed in a window of time associated with an asset. The window of time could be a few days, weeks, months, or years, for example. The platform 110 could also identify a certain number of fluid reports associated with an asset. For example, the fluid reports could be the last 3 fluid reports stored in the fluid data source 106 for the asset. The platform 110 may then receive the requested historical fluid data. Other arrangements are also possible.

At 504, the platform 110 may analyze the fluid reports to determine relationships between (1) properties of a fluid report and (2) whether the asset has a fluid issue as indicated by a fluid report. For example, each of the fluid reports obtained at 502 may indicate whether the asset associated with the fluid report has a fluid issue. The platform 110 may analyze each of these fluid reports to determine how each property in the fluid report affects whether the asset has a fluid issue. In some embodiments, results of this analysis may be captured in a variable importance statistic. The variable importance statistic may characterize how one or more given properties of a fluid influences the likelihood of an asset having a fluid issue.

At 506, the relationships for each of the properties of a fluid, may be combined together to define a predictive model for predicting an overall likelihood of an asset having a fluid issue based on properties of a fluid indicated by a fluid report or plurality of fluid reports.

In this regard, the predictive model that is defined may depend on the specific historical fluid data that is used to train the model. The historical fluid data used to define the model may include all or some subset of the fluid reports for a specific asset, all or some subset of the fluid reports for a certain type of fluid, all or some subset of the fluid reports for a certain type of asset, or all or some subset of the fluid reports for a certain subsystem of an asset. The subset could be those fluid reports based on an analysis conducted within a window of time, those fluid reports based on a fluid sampled within a window of time, or a certain number of fluid reports. Other arrangements are also possible.

For example, the historical fluid data could include one or more fluid reports associated with an asset where there is no fluid issue and one or more fluid reports which indicate that there is a fluid issue. The fluid reports indicating no fluid issue could be based on fluid samples taken at some time in the past and the fluid report which indicates a fluid issue could be based on a most recent sample of fluid. As another example, the historical fluid data may include only one or more fluid reports associated with one or more assets where there is no fluid issue. As yet another example, the historical fluid data may only include one or more fluid reports associated with one or more assets having a fluid issue.

In some examples, the historical fluid data may include fluid reports indicative of changes in the properties of the fluid over time. For instance, the historical fluid data may include a plurality of fluid reports associated with samples of fluid taken over time which show how the properties of the fluid change over time. The plurality of fluid reports could include the last N fluid reports generated for an asset, the last fluid report associated with a fluid issue for the asset and one or more previous fluid reports which are not associated with a fluid issue, or all of the fluid reports for the asset over a certain window of time. In other examples, the historical fluid data might also include a relative difference of properties of the fluid compared to other similar or different assets.

In example implementations, the platform 110 may utilize one or more modeling techniques that define a model which returns a probability between zero and one which indicates the likelihood of the asset having a fluid issue. The modeling techniques may include a random forest technique, logistic regression technique, among other regression or classification techniques to define the model. The modeling techniques may consider the properties of the fluid for each of the fluid reports an indication of whether the fluid report indicates a fluid issue. In some embodiments, a subset of historical data may be used for defining the predictive model and a mutually exclusive subset may be used to test or validate the predictive model.

In addition, during the modeling phase, the platform 110 may also define a threshold probability above which an asset may have a fluid issue as determined by the predictive model during the asset monitoring phase 404. Alternatively, the definition of the threshold could be done offline via human intervention. In either case, the threshold may be based on the one or more of historical fluid reports, indications of whether there is fluid issue, and performance requirements of the model in terms of model performance metrics such as accuracy, precision, recall, or area-under-the-curve.

The platform 110 may could also store, with the model, data associated with the fluid reports used to generate the model. The data may be stored in a data record. For instance, if the historical fluid data used to generate the model were all associated with a same type of the fluid, then this type may be stored with the model. As another example, if the historical fluid data used to generate the model were all associated with a particular source or sources (e.g., asset type or subsystem of an asset), then this source or these sources may be stored with the model.

As the fluid data source 106 continues to receive fluid reports and indications of whether each such fluid report indicates a fluid issue, the platform 110 may continue to refine the model by repeating blocks 502-506 using updated information. Further, the platform 110 may develop separate models corresponding to various types of fluids or various types of assets, for example. This way the platform 110 may build a database of models for predicting the probability of an asset having a fluid issue for a type of fluid or type of asset. In some embodiments, the platform 110 may define a single model which may be a combination of the separate models. These model(s) may be shared with other platforms 110 of the system configuration. Alternatively, each model may be an ensemble of several individual models that predict likelihood of fluid issue, which are then aggregated to predict the final likelihood.

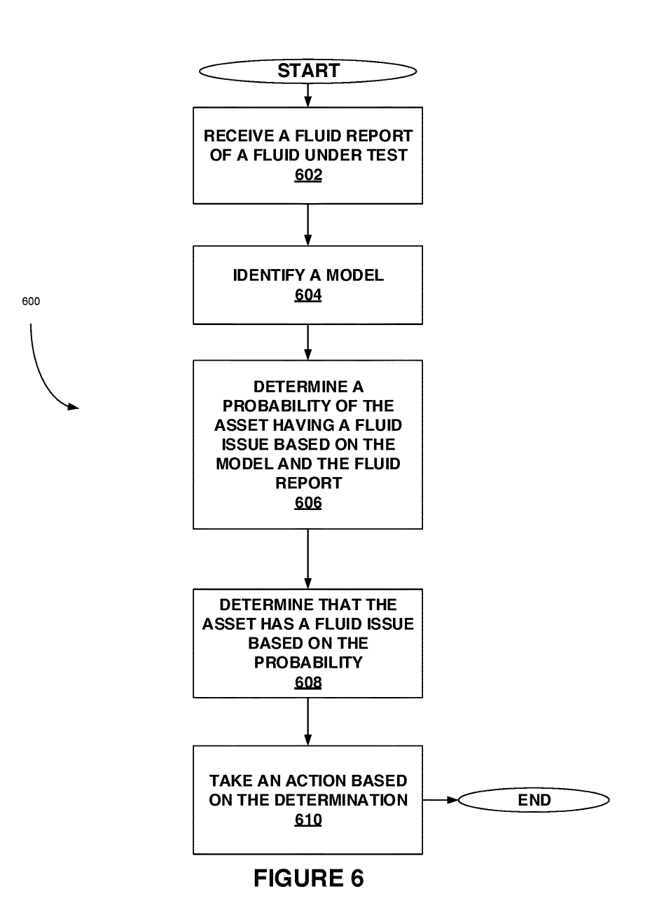

The functions of the example “asset-monitoring” phase illustrated in FIG. 6 will now be described in further detail. FIG. 6 is a flow diagram 600 depicting one possible example of an asset-monitoring phase that may be used in determining the probability of an asset having a fluid issue based on a fluid analysis. The platform 110 may begin at 602, by receiving from the fluid data source 106 a fluid report of a fluid under test from an asset. At 604, a predictive model is identified. At 606, a determination is made of a probability of the asset having a fluid issue based on the model and the fluid report. At 608, a determination is made that the asset has a fluid issue based on the probability. At 608, an action is taken based on the determination.

The functions of the example asset monitoring phase illustrated in FIG. 6 will now be described in further detail.

At 602, the platform 110 may receive from the fluid data source 106 a fluid report of a “fluid under test.” This may be a fluid from an asset where it is not known whether the asset has a fluid issue. In some embodiments, the fluid report may be a plurality of fluid reports associated with recent fluid analysis of the fluid of the asset taken at different times (e.g., a sequence of fluid reports). At 604, the platform 110 may identify a predictive model. In the case that the platform has a plurality of models, the model that is identified may be based on data provided with the fluid report such as a type of fluid being sampled, a type of asset sourcing the fluid, a specific asset, or a type of subsystem. The platform 110 may use the data provided with the fluid report to identify the appropriate model.

At 606, the platform 110 may determine a probability of the asset having a fluid issue based on the model and the fluid report. In this regard, the platform 110 may apply the model to the fluid report of the fluid under test which in turn results in a probability that the asset has a fluid issue. For example, the fluid report may define properties of the fluid. The model may take these properties as input and determine a probability of whether the asset associated with the fluid report has a fluid issue. Additionally, or alternatively, the model may take as input a plurality of fluid reports which enables the model to also consider changes in the fluid properties over time in determining the probability of the asset having a fluid issue.

At 608, the platform 110 may determine that the asset has a fluid issue based on the probability. For example, the platform 110 may compare the probability output by the model to a threshold. For instance, if the probability is greater than or equal to the threshold determined during the modeling phase then the platform 110 may identify that the asset has a fluid issue. If the probability is less that the threshold determined during the modeling phase, the platform 110 might not identify that the asset has a fluid issue.

At 610, the platform 110 may take an action based on the determination that the asset has a fluid issue. In one example, the platform may cause a computing device, such as the client station 112, to output an indication of the determination. The indication of the determination may be an audible indication and/or visual indication on the user interface of the client station 112 associated with the platform 110. The indication may be a warning or alert, e.g., email, pop-up message, or alarm, to a user of the client station 112 indicating that the asset has the fluid issue.

In another example, the platform 110 may facilitate creating a work order to repair the asset. The platform 110 may transmit work-order data to a work-order system that causes the work-order system to output a work order. The work order may specify a certain repair to the asset to alleviate the fluid issue. Additionally, or alternatively, the platform 110 may cause the computing device to present an indication of the work order on the client station 112 and even may allow a user of the client station 112 to authorize a work order prior to it being executed.

In yet another example, the platform 110 may facilitate generating and sending part-order data. The part-order data may cause a parts-ordering system to order a particular part for the asset. The particular part may be used to repair the asset to alleviate the fluid issue.

In another example, the platform 110 may transmit to the asset one or more commands that facilitate modifying one or more operating conditions of the asset to reduce chances for damage to the asset until the fluid issue is addressed. For instance, a command may cause the asset to decrease (or increase) velocity, acceleration, fan speed, propeller angle, and/or air intake among other examples.

In embodiments, the model can also be used to identify which properties of a fluid may influence the probability of the asset having a fluid issue. In general, the platform 110 may select a particular property in the fluid report and vary a measure associated with the property to determine how the variation affects the probability of the asset having the fluid issue. The platform 110 may repeat this process for one or more properties in the fluid report to determine the one or more properties of the fluid report which may influence the probability of the asset having a fluid issue.

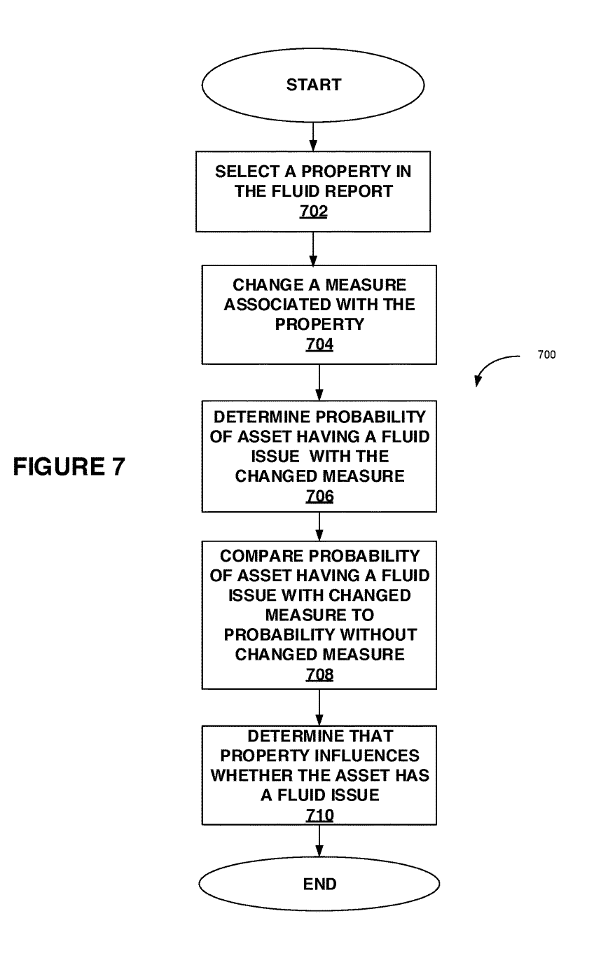

FIG. 7 is a flow diagram 700 which depicts this process in more detail. At 702, a property in the fluid report may be selected. This selection may be performed in various manners. In one implementation, the selection may be based on the property measurements in the fluid report. For instance, the platform 110 may look at particle counts and select the property that has the highest particle count measure. Other approaches for selecting a property are possible as well.

At 704, the measure of the selected property in the fluid report may be changed. In some embodiments, the amount the measure is changed may depend on the type of property. For instance, certain properties may be present in large quantities in the fluid so the change in measure may be large. In the example of particle count, the change could span several hundred ppm. On the other hand, properties may be present in small quantities in the fluid, so the change in measure may be small. In the example of particle count, the change could span only 10 ppm.

At 706, the model may be used to determine the probability of the asset having a fluid issue based on the changed measure. For example, the measure may be increased or decreased from that in the fluid report and the fluid report with the changed measure may be input into the model. The model may output the probability of the asset having a fluid issue with the changed measure.

At 708, the probability of the asset having a fluid issue as a result of the changed measure may be compared to the probability of the asset having a fluid issue without the changed measure. For example, a difference between the probability of the asset having a fluid issue based the fluid report with the changed measure and without the changed measure may be calculated.

At 710, a determination is made that the property influences whether the asset has a fluid issue. For example, the difference in probability may be compared to a threshold amount. This threshold amount may be defined by a user through the client station 112, be a predetermined value, or determined during the modeling phase. If the probability exceeds this threshold amount, then the property may influence whether the asset has a fluid issue. In other words, variations in this property may affect whether a fluid issue exists for an asset.

Once this determination is made, the platform 110 may then repeat steps 702-710 for other properties of the fluid report. This process may continue until each one of the properties is analyzed and the process ends.

In some embodiments, the platform 110 may also present a user with an identification of the one or more properties that influence the likelihood of a fluid issue. For example, the platform 110 may cause an indication of these one or more properties to be presented on the user interface of the client station 112. In this regard, a user may be notified of the particular properties of the fluid report which are relevant to the determination of whether the asset has a fluid issue. These particular properties may be highlighted on the client station so that the user may be able to focus attention on the particular properties for purposes of asset management.

The one or more properties identified in FIG. 7 and in general based on the fluid report may also be used, for example, to specify a certain repair of the asset to alleviate the fluid issue. For example, the platform 110 may have a database of recommended repairs that are correlated to the properties of the fluid. The platform 110 may input the properties of the fluid into the database and the database may identify the specific repair of the asset to alleviate the fluid issue. As another example, the platform 110 may input the property or properties influencing the probability of the asset having a fluid issue, and the database may identify the specific repair of the asset to alleviate the fluid issue. Other arrangements are also possible.

Additionally, the platform 110 may present information associated with the fluid report or plurality of fluid reports. The information may be presented as a graphical user interface (GUI) on the client station 112. For instance, in one implementation, the GUI may include a textual or graphical representation of particles counts for the various particles in a given type of fluid taken from a given asset. In another implementation, the GUI may include a textual and/or graphical presentation of how certain properties of the given type of fluid taken from the given asset compare to properties of the given type of fluid taken from other assets or from the same asset in the past.

In yet another implementation, the GUI may indicate how the particle counts in a fluid of an asset may vary over time for the asset. For example, the GUI may indicate how a count of a particular particle in the fluid of an asset changes over weeks, months, or years. In another example, the GUI may compare properties of a fluid of an asset to properties of a same fluid from other assets based on the historical fluid data. The comparison may be based on average particle counts for assets of a same type. In another example, the GUI may identify one or more of those properties that influence whether the asset has a fluid issue. In yet another example. the GUI may further identify the measure of those properties which influence whether the asset has a fluid issue and/or optimal measures for those properties. This information may be presented numerically or in the form a graph such as bar graph or line graph on the GUI.

In another implementation, the GUI may show fluid properties such as age of the fluid, viscosity, pH and such and/or the fluid issue that was determined by the predictive model. The fluid issue could be represented as a likelihood of an asset having a fluid issue or a class of a fluid issue such as “contamination”, “leak”, “wear and tear of components”. In another embodiment, the GUI may show possible actions required such as work order to be created, maintenance procedures to be performed and so on.

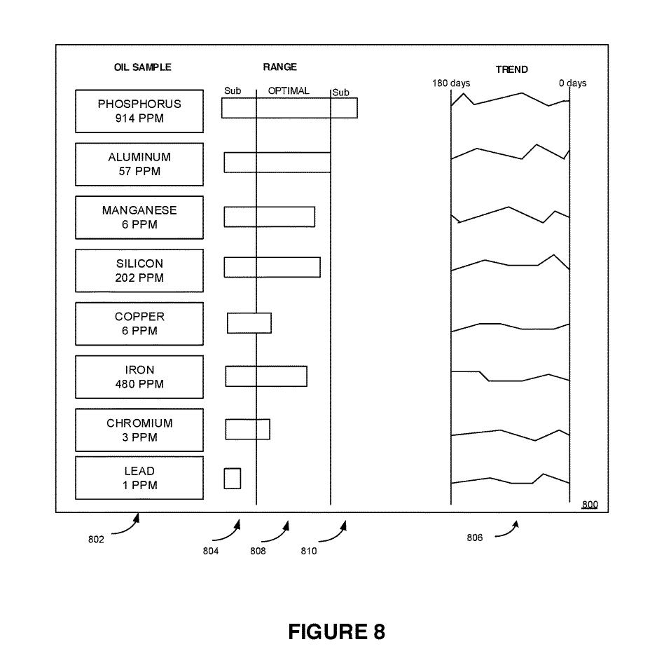

FIG. 8 illustrates an example GUI 800. The GUI 800 illustrates various properties 802 in a fluid report of oil, such as amounts of phosphorus, aluminum, manganese, silicon, copper, iron, chromium, and lead, and specifically the particle count of these properties in ppm. In some embodiments, the properties shown may be those which influence whether an asset has a fluid issue. Additionally, the GUI 800 shows ranges 804, 808, 810 of particle counts in the form of a bar graph. The bar graph may be normalized so as to scale measures of properties into one of the ranges 804, 808, 810. The range 808 may be an optimal range, and the ranges 804 and 810 may be suboptimal range, either with particle counts below the optimal range in range 804 or particle counts above the optimal range in range 810. In the example GUI 800, the particle count for phosphorus exceeds the optimal range 808. Also, in the example GUI 800, the particle count for lead is less than the optimal range 808. Particle counts in the suboptimal ranges may or might not indicate that the asset has a fluid issue. Still additionally, the GUI 800 may show trends 806 in each of the properties. For instance, the GUI may show how the particle count for each property may change over time such as over the last 180 days. This may be represented as a line graph where the amplitude of the line graph is indicative of the particle count at a particular time.

### IV. Example Method

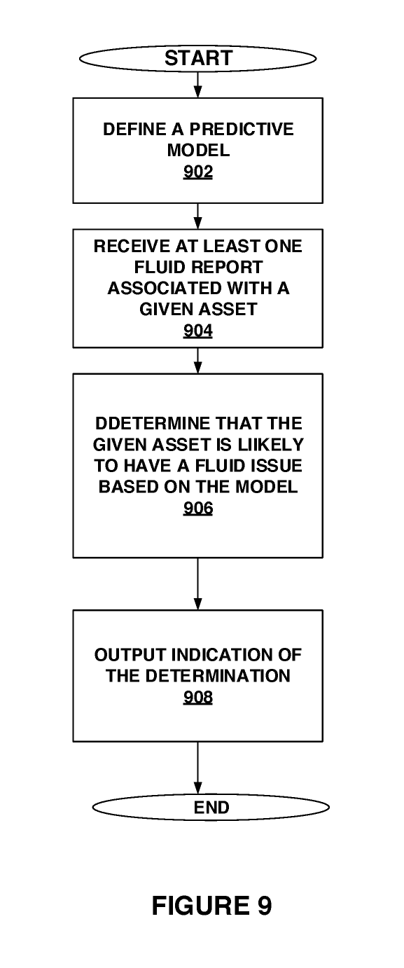

Turning now to FIG. 9, a flow diagram is depicted illustrating an example method 900 for determining that an asset is likely to have a fluid issue. For the method 900, the operations illustrated by the blocks in the flow diagrams may be performed in line with the above discussion. Moreover, one or more operations discussed above may be added to a given flow diagram.

At 902, a predictive model is defined for outputting an indicator of whether an asset is likely to have a fluid issue. The predictive model may be based at least on historical fluid data for one or more assets. Further, the historical fluid data may comprise at least one of (i) a plurality of fluid reports associated with the one or more assets and (ii) a plurality of indications, where each indication identifies whether an asset of the one or more assets has a fluid issue. The predictive model may be defined by the platform 110, and/or with human intervention, or exclusively by a human, for example. Further the indication of whether the asset has a fluid issue may be provided by an expert and/or by a computer system.

At 904, at least one fluid report associated with a given asset is received. For example, the fluid report may be received from a fluid data source 106. At 906, a determination is made that the given asset is likely to have a fluid issue. This determination may be made based at least on the predictive model and the received at least one fluid report. At 908, a computing device may output an indication of the determination. The indication may be output, for example, on a graphical user interface of a client station 112.

The description above discloses, among other things, various example systems, methods, apparatus, and articles of manufacture including, among other components, firmware and/or software executed on hardware. It is understood that such examples are merely illustrative and should not be considered as limiting. For example, it is contemplated that any or all of the firmware, hardware, and/or software aspects or components can be embodied exclusively in hardware, exclusively in software, exclusively in firmware, or in any combination of hardware, software, and/or firmware. Accordingly, the examples provided may not be the only way(s) to implement such systems, methods, apparatus, and/or articles of manufacture.

Additionally, references herein to “embodiment” means that a particular feature, structure, or characteristic described in connection with the embodiment can be included in at least one example embodiment of an invention. The appearances of this phrase in various places in the specification are not necessarily all referring to the same embodiment, nor are separate or alternative embodiments mutually exclusive of other embodiments. As such, the embodiments described herein, explicitly and implicitly understood by one skilled in the art, can be combined with other embodiments.

The specification is presented largely in terms of illustrative environments, systems, procedures, steps, logic blocks, processing, and other symbolic representations that directly or indirectly resemble the operations of data processing devices coupled to networks. These process descriptions and representations are typically used by those skilled in the art to most effectively convey the substance of their work to others skilled in the art. Numerous specific details are set forth to provide a thorough understanding of the present disclosure. However, it is understood to those skilled in the art that certain embodiments of the present disclosure can be practiced without certain, specific details. In other instances, well known methods, procedures, components, and circuitry have not been described in detail to avoid unnecessarily obscuring aspects of the embodiments. Accordingly, the scope of the present disclosure is defined by the appended claims rather than the forgoing description of embodiments.

When any of the appended claims are read to cover a purely software and/or firmware implementation, at least one of the elements in at least one example is hereby expressly defined to include a tangible, non-transitory medium such as a memory, DVD, CD, Blu-ray, and so on, storing the software and/or firmware.

To the extent that examples described herein involve operations performed or initiated by actors, such as “humans”, “operators”, “users” or other entities, this is for purposes of example and explanation only. Further, the term “fluid” may refer to one type of fluid or multiple types of fluid. Moreover, the claims should not be construed as requiring action by such actors unless explicitly recited in the claim language.

We claim:

1. A method comprising:
based at least on historical fluid data for one or more assets, defining a predictive model for outputting an indicator of whether an asset is likely to have a fluid issue, wherein the historical fluid data comprises at least one of (i) a plurality of fluid reports associated with the one or more assets and (ii) a plurality of indications, wherein each indication identifies whether an asset of the one or more assets has a fluid issue;
receiving at least one fluid report associated with a given asset;
based at least on the predictive model and the received at least one fluid report, making a determination that the given asset is likely to have a fluid issue; and
causing a computing device to output an indication of the determination.

2. The method of claim 1, wherein the indication of a fluid issue is determined based on an expert review of the fluid report.

3. The method of claim 1, wherein defining the predictive model comprises applying a regression technique to the historical fluid data.

4. The method of claim 1, wherein the historical fluid data comprises one or more of (i) a plurality of fluid reports for a same type of fluid; (ii) a plurality of fluid reports for a same type of the given asset; (iii) a plurality of fluid reports for the given asset; and (iv) a plurality of fluid reports for a same type of subsystem of the given asset.

5. The method of claim 1, wherein each of the fluid reports comprises a plurality of fluid properties.

6. The method of claim 5, wherein the fluid properties comprises a particle count of particles in a fluid.

7. The method of claim 1, wherein each fluid report is for a given type of fluid taken from the given asset.

8. The method of claim 1, wherein the plurality of fluid reports comprises a sequence of fluid reports for the given asset.

9. The method of claim 1, wherein causing a computing device to output an indication of the determination comprises causing a computing device to display the determination on a display screen.

10. The method of claim 1, wherein making a determination that the given asset is likely to have a fluid issue based at least on the predictive model and the received at least one fluid report comprises:
applying the predictive model to the received at least one fluid report to output an indicator of whether the given asset is likely to have a fluid issue;
comparing the indicator to a threshold condition; and
determining that the indicator exceeds the threshold condition.

11. The method of claim 1, further comprising causing the computing device to output an indication of those properties of a given type of fluid of the given asset which influence the determination that the given asset is likely to have the fluid issue.

12. The method of claim 1, wherein the received at least one fluid report defines a plurality of properties of a fluid and a measure associated with each property, the method further comprising:
selecting a given property of the received at least one fluid report;
changing a given measure of the given property in the at least one fluid report;
making a determination that changing the given measure causes a probability that the given asset has the fluid issue to change by a threshold amount; and
causing the computing device to output an indication of the given property.

13. A platform comprising:
a network interface configured to facilitate communication with a data source and a computing device via a communication network;
at least one processor;
a non-transitory computer-readable medium; and
program instructions stored on the non-transitory computer-readable medium that are executable by the at least one processor to cause the platform to:
receive at least one fluid report associated with a given asset;
based at least on a predictive model which outputs an indicator of whether an asset is likely to have a fluid issue and the received at least one fluid report, make a determination that the given asset is likely to have a fluid issue, wherein the predictive model is defined based at least on historical fluid data for one or more assets received from the data source, wherein the historical fluid data comprises at least one of (i) a plurality of fluid reports associated with the one or more assets and (ii) a plurality of indications, wherein each indication identifies whether an asset of the one or more assets has a fluid issue; and
cause the computing device to output an indication of the determination.

14. The platform of claim 13, wherein the plurality of fluid reports comprises a time sequence of fluid reports for the given asset.

15. The platform of claim 13, wherein the program instructions for causing a computing device to output an indication of the determination comprises causing the computing device to display the determination on a display screen.

16. The platform of claim 13, wherein the historical fluid data comprises one or more of (i) a plurality of fluid reports for a same type of fluid; (ii) a plurality of fluid reports for a same type of the given asset; (iii) a plurality of fluid reports for the given asset; and (iv) a plurality of fluid reports for a same type of subsystem of the given asset.

17. The platform of claim 13, wherein the program instructions for making a determination that the given asset is likely to have a fluid issue based at least on the predictive model and the received at least one fluid report comprises program instructions for:
applying the predictive model to the received at least one fluid report to output an indicator of whether the given asset is likely to have a fluid issue;
comparing the indicator to a threshold condition; and
determining that the indicator exceeds the threshold condition.

18. The platform of claim 13, further comprising program instructions for causing the computing device to output an indication of how properties of a given type of fluid in the given asset compare to properties of the given type of fluid in other assets based on the historical fluid data.

19. The platform of claim 13, wherein the received at least one fluid report defines a plurality of properties of a fluid and a measure associated with each property, the platform further comprising program instructions for:
selecting a given property of the received at least one fluid report;
changing a given measure of the given property in the at least one fluid report;
making a determination that changing the given measure causes a probability that the given asset has the fluid issue to change by a threshold amount; and
causing the computing device to output an indication of the given property.

20. A non-transitory computer-readable medium having instructions stored thereon that are executable to cause a platform to:
receive at least one fluid report associated with a given asset;
based at least on a predictive model which outputs an indicator of whether an asset is likely to have a fluid issue and the received at least one fluid report, make a determination that the given asset is likely to have a fluid issue, wherein the predictive model is defined based at least on historical fluid data for one or more assets received from a data source, wherein the historical fluid data comprises at least one of (i) a plurality of fluid reports associated with the one or more assets and (ii) a plurality of indications, wherein each indication identifies whether an asset of the one or more assets has a fluid issue; and
cause a computing device to output an indication of the determination.
